(19) United States
(12) Patent Application Publication
(10) Pub. No.: US 2026/0206794 A1
(43) Pub. Date: Jul. 23, 2026

(54) PROCESS FOR REDUCING SYRUP VISCOSITY IN THE BACKEND OF A PROCESS FOR PRODUCING A FERMENTATION PRODUCT

(71) Applicant: Novozymes A/S, Bagsvaerd (DK)

(72) Inventors: Jiyin LIU, Raleigh, NC (US); Hui XU, Wake Forest, NC (US); Melissa Carrie HOOSS, Franklinton, NC (US)

(73) Assignee: Novozymes A/S, Bagsvaerd (DK)

(21) Appl. No.: 19/137,497

(22) PCT Filed: Dec. 11, 2023
(86) PCT No.: PCT/US2023/083357

§ 371 Date: Jun. 10, 2025

Related U.S. Application Data
(60) Provisional application No. 63/476,090, filed on Dec. 19, 2022.

(51) Int. Cl. A23K 10/38; C12N 9/02; C12N 9/08; C12N 9/26; C12N 9/34

(52) U.S. Cl. CPC A23K 10/38; C12N 9/0061; C12N 9/0065; C12N 9/2414; C12N 9/2428; C12Y 110/03002; C12Y 111/01007; C12Y 302/01001; C12Y 302/01003

(57) ABSTRACT

The present invention relates to a process for reducing syrup viscosity at the backend of a process for producing a fermentation product (e.g., ethanol from corn), comprising adding an oxidoreductase after the fermenting step, before whole stillage is subject to separation, to decrease the quantity of particles in the thin stillage by increasing the size of 5 particles in the whole stillage, thereby reducing the viscosity of syrup produced by evaporating the thin stillage.

### REFERENCE TO A SEQUENCE LISTING

This application contains a Sequence Listing in computer readable form, which is incorporated herein by reference.

### FIELD OF THE INVENTION

The present invention relates to a process for reducing syrup viscosity at the backend of a process for producing a fermentation product (e.g., ethanol from corn), comprising adding an oxidoreductase after the fermenting step, before whole stillage is subject to separation, to decrease the quantity of particles in the thin stillage by increasing the size of particles in the whole stillage, thereby reducing the viscosity of syrup produced by evaporating the thin stillage.

### BACKGROUND OF THE INVENTION

In a process for producing ethanol from corn, following SSF or the RSH process, the liquid fermentation products are recovered from the fermented mash (often referred to as “beer mash”), e.g., by distillation, which separates the desired fermentation product, e.g. ethanol, from other liquids and/or solids. The remaining fraction is referred to as “whole stillage”. Whole stillage typically contains about 10 to 20% solids. The whole stillage is separated into a solid and a liquid fraction, e.g., by centrifugation. The separated solid fraction is referred to as “wet cake” (or “wet grains”) and the separated liquid fraction is referred to as “thin stillage”. Wet cake and thin stillage contain about 35 and 7% solids, respectively. Wet cake, with optional additional dewatering, is used as a component in animal feed or is dried to provide “Distillers Dried Grains” (DDG) used as a component in animal feed. Thin stillage is typically evaporated to provide evaporator condensate and syrup or may alternatively be recycled to the slurry tank as “backset”. Evaporator condensate may either be forwarded to a methanator before being discharged and/or may be recycled to the slurry tank as “cook water”. The syrup may be blended into DDG or added to the wet cake before or during the drying process, which can comprise one or more dryers in sequence, to produce DDGS (Distillers Dried Grain with Solubles). Syrup typically contains about 25% to 35% solids. Oil can also be extracted from the thin stillage and/or syrup as a by-product for use in biodiesel production, as a feed or food additive or product, or other biorenewable products.

Although decanting of whole stillage removes large particles into wet cake, there are still smaller particles and soluble nutrients in thin stillage. Small particles in thin stillage contain valuable materials such as proteins, lipids and carbohydrates. Small particles including insoluble solids and some soluble nutrients are typically concentrated into syrup through a multiple-step (e.g. seven evaporation tankers) high temperature evaporation process. As water is evaporated to concentrate the thin stillage into syrup, small particles in thin stillage become increasingly concentrated, which increases syrup viscosity.

Though improvements in the efficiency of corn kernel milling, chemical hydrolysis, and enzymatic hydrolysis have helped to maximize the conversion of starch to ethanol, they have also resulted in greater amounts of small particles that concentrate in the thin stillage after decanting, further increasing syrup viscosity to potentially problematic levels. Therefore, there is a need for improved processes for reducing syrup viscosity in the back end of commercial ethanol plants.

### SUMMARY OF THE INVENTION

The present invention provides a solution to the above problem by providing a process for reducing syrup viscosity at the backend of a process for producing a fermentation product (e.g., ethanol from corn), comprising adding an oxidoreductase after the fermenting step, before whole stillage is subject to separation, to decrease the quantity of particles in the thin stillage by increasing the size of particles in the whole stillage, thereby reducing the viscosity of syrup produced by evaporating the thin stillage.

Accordingly, in an aspect, a process for reducing the viscosity of syrup in a process for producing a fermentation product from a starch-containing material comprises:
(a) liquefying a starch-containing material with a thermostable alpha-amylase at a temperature above the initial gelatinization temperature of the starch to produce a dextrin;
(b) saccharifying the dextrin with a glucoamylase to produce a fermentable sugar;
(c) fermenting the sugar with a fermenting organism to produce a beer comprising the fermentation product;
(d) distilling the beer to recover the fermentation product and produce a whole stillage;
(e) processing the whole stillage to produce a thin stillage; and
(f) evaporating the thin stillage to produce a syrup;
wherein an oxidoreductase is added after the fermenting step to decrease the quantity of particles in the thin stillage by increasing the size of particles in the whole stillage, thereby reducing the viscosity of syrup produced by evaporating the thin stillage.

In an aspect, a process for reducing viscosity of syrup in a producing a fermentation product from a starch-containing material comprises:
(a) saccharifying a starch-containing material at a temperature below the initial gelatinization temperature of the starch with an alpha-amylase and a glucoamylase to produce fermentable a sugar;
(b) fermenting the sugar with a fermenting organism to produce a beer comprising the fermentation product;
(c) distilling the beer to recover the fermentation product and to produce a whole stillage;
(d) processing the whole stillage to produce a thin stillage; and
(e) evaporating the thin stillage to produce a syrup;
wherein an oxidoreductase is added after the fermenting step to decrease the quantity of particles in the thin stillage by increasing the size of particles in the whole stillage, thereby reducing the viscosity of syrup produced by evaporating the syrup.

The oxidoreductase may be added to the beer well containing the beer prior to the distilling step, during the distilling step, or to the whole stillage before the processing step.

The processing step may include a separation step performed by subjecting the whole stillage to a filtration centrifuge, a decanter centrifuge, a pressure screen, or a paddle screen. In an embodiment, the centrifuge is a decanter centrifuge, particularly a horizontal decanter centrifuge.

The oxidoreductase may be added to the whole stillage and incubated for a period of time before the whole stillage is subjected to the separation step.

The oxidoreductase may be used at a temperature in the range from 30° C. to 100° C., such as 40° C. to 90° C., preferably 45° C. to 85° C.

The cumulative passing is reduced by from 10 volume % to 80 volume % for particles having a size ranging from 100 μm to 1000 μm.

The cumulative passing of particles having a diameter equal to 400 μm is decreased by 10 volume % to 80 volume %.

In an embodiment, the oxidoreductase is a laccase.

In an embodiment, the oxidoreductase is a peroxidase.

The saccharifying and fermenting steps may be performed simultaneously.

The starch-containing material may include beets, maize, corn, wheat, rye, barley, oats, triticale, sorghum, sweet potatoes, rice, millet, pearl millet, and/or foxtail millet.

In an embodiment, the starch-containing material comprises corn.

In an embodiment, the fermentation product comprises ethanol, preferably fuel ethanol.

In an embodiment, the fermenting organism is yeast.

### BRIEF DESCRIPTION OF THE FIGURES

FIG. 1 shows that the particle diameter in thin stillage is under 400 μm, while whole stillage has particle diameter in a range of 0 to 1820 μm.

FIG. 2 shows the impact on cumulative passing of whole stillage teated with Oxidoreductase B with and without hydrogen peroxide at 65° C.

FIG. 3 shows the impact on cumulative passing of whole stillage teated with Oxidoreductase A and Oxidoreductase B at 65° C.

FIG. 4 shows the impact on cumulative passing of whole stillage teated with Oxidoreductase A and Oxidoreductase B at 50° C.

FIG. 5 shows the impact on cumulative passing of whole stillage teated with Oxidoreductase A and Oxidoreductase B at 35° C.

### DEFINITIONS

Auxiliary Activity 1 (AA1) Family: AA1 Family includes enzymes characterized as multicopper oxidases that use diphenols and related substances as donors with oxygen as the acceptor. The AA1 family is divided into three subfamilies, including laccases, ferroxidases and laccase-like multicopper oxidases.

Auxiliary Activity 2 (AA2) Family: AA2 Family contains class II lignin-modifying peroxidases. AA2 enzymes are secreted heme-containing enzymes that use hydrogen peroxidase or organic peroxides as electron acceptors to catalyze a variety of oxidative reactions where two electrons are derived from substrates to reduce the enzyme with the concomitant release of two water molecules.

Cumulative Passing: “Cumulative passing” means the volume percentage of particles less than or equal to a particular size which passes through the whole stillage separation into the thin stillage.

Fermentation product: “Fermentation product” means a product produced by a process including fermenting using a fermenting organism. Fermentation products include alcohols (e.g., ethanol, methanol, butanol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. In an embodiment the fermentation product is ethanol.

Fermenting organism: “Fermenting organism” refers to any organism, including bacterial and fungal organisms, especially yeast, suitable for use in a fermentation process and capable of producing the desired fermentation product.

GH3 beta-xylosidase: “GH3 beta-xylosidase” is an abbreviation for Glycoside Hydrolase Family 3 beta-xylosidases, which are xylan 1,4-beta-xylosidases (EC 3.2.1.37) that catalyze the hydrolysis (1→4)-β-D-xylans, to remove successive D-xylose residues from the non-reducing termini.

Initial gelatinization temperature: “Initial gelatinization temperature” means the lowest temperature at which gelatinization of the starch commences. Starch heated in water begins to gelatinize between 50 degrees centigrade and 75 degrees C.; the exact temperature of gelatinization depends on the specific starch, and can readily be determined by the skilled artisan. Thus, the initial gelatinization temperature may vary according to the plant species, to the particular variety of the plant species as well as with the growth conditions. In the context of this disclosure the initial gelatinization temperature of a given starch-containing material is the temperature at which birefringence is lost in 5 percent of the starch granules using the method described by Gorinstein. S. and Lii. C, Starch/Starke, Vol. 44 (12) pp. 461-466 (1992).

Laccase: “Laccase” refers to a benzenediol:oxygen oxidoreductase (EC 1.10.3.2) that catalyzes the reaction of 4 benzendiol+O₂=4 benzosemiquinone+2 H₂O. These laccases include multi-copper proteins of low specificity acting on both o- and p-quinols, and often acting on aminophenols and phenylenediamine.

Laccase Activity: Laccase (EC 1.10.3.2) activity can be assayed based on its ability to catalyze oxidation of syringaldazine (4,4′-[azinobis(methanylylidene)]bis(2,6-dimethoxyphenol)) to the corresponding quinone 4,4′-[azobis(methanylylidene])bis(2,6-dimethoxycyclohexa-2,5-dien-1-one), see Formula 1 below.

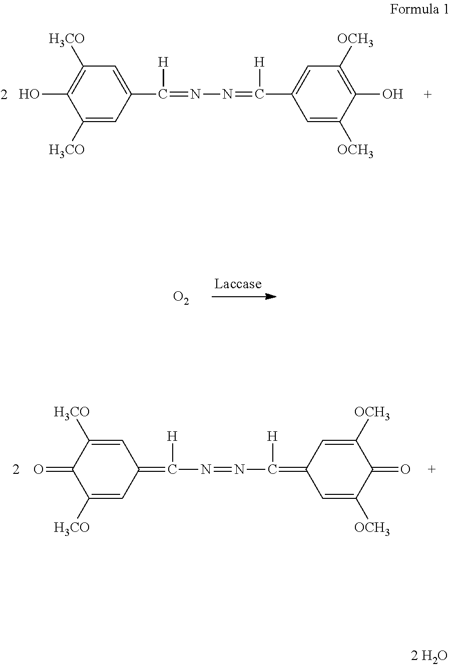 [SMILES: COc1cc(C=NN=Cc2cc(OC)c(O)c(OC)c2)cc(OC)c1O ; "O2" ; COC1=CC(=CN=NC=C2C=C(OC)C(=O)C(OC)=C2)C=C(OC)C1=O]

The reaction is detected by the increase in absorbance at 530 nm.

One laccase unit is the amount of enzyme which under the given analytical conditions catalyzes the conversion of 1 mmole syringaldazine per minute. For measurements made at pH 5.5 the activity units are labelled LACU, and for measurements made at pH 7.5 the activity units are labelled LAMU.

Oxidoreductase: “Oxidoreductase” refers to an enzyme that catalyzes the transfer of electrons from an electron donor (reductant) to an electron acceptor (oxidant) in a redox reaction. “Oxidoreductase” encompasses any such enzyme that is capable of increasing the volume % of larger sized particles in whole stillage, including for example, oxidoreductases (EC 1.10) acting on phenols and related substances as donors and oxidoreductases (EC 1.11) acting on peroxide as an acceptor.

Peroxidase: “Peroxidase” refers to a phenolic donor:hydrogen-peroxide oxidoreductase (EC 1.11.1.7) that catalyzes the reaction of 2 phenolic donor+H₂O₂=2 phenoxyl radical of the donor+2 H₂O. These peroxidases include heme proteins with histidine as a proximal ligand in which the iron in the resting enzyme is Fe(Ill).

Peroxidase Activity: Peroxidase (EC 1.11.1.7) activity can be assayed based on its ability to catalyze, in the presence of hydrogen peroxide, the oxidation of 2,2′-azino-di-[3-ethylbenzthiazoline-6-sulphonate] (ABTS®) to form a blue-green color. The reaction is detected by measuring the increase in absorbance at 418 nm, as shown in Formula 2 below depicting the oxidation of ABTS®. catalyzes the conversion of 1 mmole hydrogen peroxide per minute.

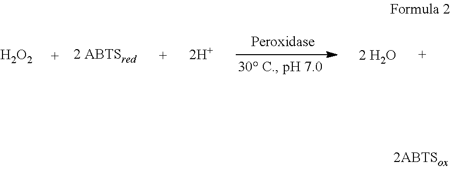

One peroxidase unit (POXU) is the amount of enzyme which under the given analytical conditions

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), e.g., version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), e.g., version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

Thermostable: “Thermostable” means the enzyme is not denatured or deactivated when it is used in a liquefaction step of a process of the invention. In other words, a thermostable enzyme is suitable for liquefaction if it has a denaturation temperature (Td) that is compatible with the liquefaction temperature and retains its activity at that temperature.

Thin Stillage: “Thin Stillage” refers to centrate separated from whole stillage that is pumped toward the evaporators to be concentrated into syrup.

Whole Stillage: “Whole stillage” includes the material that remains at the end of the distillation process after recovery of the fermentation product, e.g., ethanol.

### DESCRIPTION OF THE INVENTION

The present invention relates to a process for reducing syrup viscosity at the backend of a process for producing a fermentation product (e.g., ethanol from corn), comprising adding an oxidoreductase after the fermenting step, before whole stillage is subject to separation, to decrease the quantity of particles in the thin stillage by increasing the size of particles in the whole stillage, thereby reducing the viscosity of syrup produced by evaporating the thin stillage.

Surprisingly, work described herein demonstrates a 10 volume % to 80 volume % reduction in the cumulative passing for particles having a size ranging from 100 μm to 1000 μm. The work herein further unexpectedly demonstrates that the cumulative passing of particles having a diameter of 400 μm is decreased by 10 volume % to 80 volume %.

The significant reduction in the cumulative passing of particles having a size equal to the size that would normally go to thin stillage following decanting with the oxidoreductases (e.g., about 400 μm) indicates that oxidoreductases can reduce the viscosity of syrup, for instance, by increasing the volume % of larger size of particles in the whole stillage while decreasing the volume % of smaller sized particles in the whole stillage that would otherwise avoid decanting and accumulate in the thin stillage that is evaporated to produce the syrup.

The present invention contemplates using oxidoreductases after fermentation in conventional starch to ethanol and raw starch hydrolysis (RSH) ethanol production processes.

Process for Producing a Fermentation Product from a Gelatinized Starch-Containing Material

An aspect of the invention relates to a process for reducing syrup viscosity at the backend of a process for producing a fermentation product, (e.g., fuel ethanol), from a gelatinized starch-containing material, wherein an oxidoreductase is added after the fermenting step to decrease the quantity of particles in the thin stillage by increasing the size of particles in the whole stillage, thereby reducing the viscosity of syrup produced by evaporating the thin stillage.

In an embodiment, a process for reducing syrup viscosity (e.g., at the back-end of a process for producing a fermentation product from a starch-containing material), comprises the steps of:
(a) liquefying a starch-containing material at a temperature above the initial gelatinization temperature of the starch using a thermostable alpha-amylase to produce a dextrin;
(b) saccharifying the dextrin using a glucoamylase to produce a fermentable sugar; and
(c) fermenting the sugar using a fermenting organism to produce the fermentation product;
(d) distilling the fermentation product to recover the fermentation product and produce whole stillage;
(e) processing the whole stillage to produce a thin stillage; and
(f) evaporating the thin stillage to produce a syrup;
wherein an oxidoreductase is added after the fermenting step to decrease the quantity of particles in the thin stillage by increasing the size of particles in the whole stillage, thereby reducing the viscosity of syrup produced by evaporating the syrup.

In an embodiment, the oxidoreductase is added to a beer well containing the beer prior to the distilling step. In an embodiment, the oxidoreductase is added during the distilling step. In an embodiment, the oxidoreductase is added to the whole stillage before the processing step.

In an embodiment, the processing step comprises a separation step performed by subjecting the whole stillage to a filtration centrifuge, a decanter centrifuge, a pressure screen, or a paddle screen. In an embodiment, the centrifuge is a decanter centrifuge, particularly a horizontal decanter centrifuge.

In an embodiment, the oxidoreductase is added to the whole stillage and incubated for a period of time before the whole stillage is subjected to the separation step.

In an embodiment, the oxidoreductase is a laccase. In an embodiment, the oxidoreductase is a peroxidase.

Preferably, the oxidoreductase used in the process has an optimum temperature that is compatible with the temperature ranges of the process step in which they are used. In an embodiment, the oxidoreductase is used at a temperature in the range from 30° C. to 100° C., such as 40° C. to 90° C., preferably 45° C. to 85° C.

In an embodiment, a thermostable glucoamylase is added during liquefying step (a). In an embodiment, a thermostable endoglucanase is added during liquefying step (a). In an embodiment, a thermostable lipase is added during liquefying step (a). In an embodiment, a thermostable phytase is added during liquefying step (a). In an embodiment, a thermostable protease is added during liquefying step (a). In an embodiment, a thermostable pullulanase is added during liquefying step (a). In an embodiment, a thermostable xylanase is added during liquefying step (a). In a preferred embodiment, a thermostable alpha-amylase and a thermostable protease are added during liquefying step (a). In an embodiment, a thermostable alpha-amylase and a thermostable xylanase are added during liquefying step (a). In a preferred embodiment, a thermostable alpha-amylase, a thermostable protease and a thermostable xylanase are added during liquefying step (a).

In an embodiment, an alpha-amylase is added during step (b) and/or step (c). In an embodiment, an alpha-glucosidase is added during step (b) and/or step (c). In an embodiment, a beta-amylase is added during step (b) and/or step (c). In an embodiment, a beta-glucanase is added during step (b) and/or step (c). In an embodiment, a beta-glucosidase is added during step (b) and/or step (c). In an embodiment, a cellobiohydrolase is added during step (b) and/or step (c). In an embodiment, an endoglucanase is added during step (b) and/or step (c). In an embodiment a lipase is added during step (b) and/or step (c). In an embodiment, a lytic polysaccharide monooxygenase (LPMO) is added during step (b) and/or step (c). In an embodiment, a maltogenic alpha-amylsae is added during step (b) and/or step (c). In an embodiment, a pectinase is added during step (b) and/or step (c). In an embodiment, a peroxidase is added during step (b) and/or step (c). In an embodiment, a phytase is added during step (b) and/or step (c). In an embodiment, a protease is added during step (b) and/or step (c). In an embodiment, a trehalase is added during step (b) and/or step (c). In an embodiment, a xylanase is added during step (b) and/or step (c).

In an embodiment, the fermenting organism is yeast. In an embodiment, the yeast expresses an alpha-amylase in situ during step (b) and/or step (c). In an embodiment, the yeast expresses a glucoamylase in situ during step (b) and/or step (c).

### Process Parameters

The parameters for processes for producing fermentation products, such as the production of ethanol from starch-containing material (e.g., corn) are well known in the art. See, e.g., WO 2006/086792, WO 2013/082486, WO 2012/088303, WO 2013/055676, WO 2014/209789, WO 2014/209800, WO 2015/035914, WO 2017/112540, WO 2020/014407, WO 2021/126966 (each of which is incorporated herein by reference).

### Starch-Containing Material

Any suitable starch-containing starting material may be used. The material is selected based on the desired fermentation product. Examples of starch-containing materials, include without limitation, barley, beets, beans, cassava, cereals, corn, milo, oats peas, potatoes, rice, rye, sago, sorghum, sweet potatoes, tapioca, wheat, and whole grains, or any mixture thereof. The starch-containing material may also be a waxy or non-waxy type of corn and barley. Commonly used commercial starch-containing materials include corn, milo and/or wheat.

### Starch-Containing Material Particle Size Reduction

Prior to liquefying step (a), the particle size of the starch-containing material may be reduced, for example by dry milling.

### Slurry

Prior to liquefying step (a), a slurry comprising the starch-containing material (e.g., preferably milled) and water may be formed. Alpha-amylase and optionally protease may be added to the slurry. The slurry may be heated to between to above the initial gelatinization temperature of the starch-containing material to begin gelatinization of the starch.

### Jet Cook

The slurry may optionally be jet-cooked to further gelatinize the starch in the slurry before adding alpha-amylase during liquefying step (a). Jet cooking can be performed at temperatures ranging from 100° C. to 120° C. for up to at least 15 minutes.

### Liquefaction Temperature

The temperature used during liquefying step (a) may range from 70° C. to 110° C., such as from 75° C. to 105° C., from 80° C. to 100° C., from 85° C. to 95° C., or from 88° C. to 92° C. Preferably, the temperature is at least 70° C., at least 80° C., at least 85° C., at least 88° C., or at least 90° C.

### Liquefaction pH

The pH used during liquefying step (a) may range from 4 to 6, from 4.5 to 5.5, or from 4.8 to 5.2. Preferably, the pH is at least 4.5, at least 4.6, at least 4.7, at least 4.8, at least 4.9, at least 5.0, or at least 5.1.

### Liquefaction Time

The time for performing liquefying step (a) may range from 30 minutes to 5 hours, from 1 hour to 3 hours, or 90 minutes to 150 minutes. Preferably, the time is at least 30 minutes, at least about 45 minutes, at least about 60 minutes, at least about 90 minutes, or at least about 2 hours.

### Liquefaction Enzymes

The present invention contemplates the use of thermostable enzymes during liquefying step (a). It is well known in the art to use various thermostable enzymes during liquefying step (a), including, for example, thermostable alpha-amylases, thermostable glucoamylases, thermostable endoglucanases, thermostable lipases, thermostable phytase, thermostable proteases, thermostable pullulanases, and/or thermostable xylanases. The present invention contemplates the use of any thermostable enzyme in liquefying step (a). Guidance for determining the denaturation temperature of a candidate thermostable enzyme for use in liquefying step (a) is provided in the Materials & Methods section below. The published patent applications listed below describe activity assays for determining whether a candidate thermostable enzyme contemplated for use in liquefying step (a) will be deactivated at a temperature contemplated for liquefying step (a).

Examples of suitable thermostable alpha-amylases and guidance for using them in liquefying step (a) include, without limitation, the alpha-amylases described in WO94/18314, WO94/02597, WO 96/23873, WO 96/23874, WO 96/39528, WO 97/41213, WO 97/43424, WO 99/19467, WO 00/60059, WO 2002/010355, WO 2002/092797, WO 2009/149130, WO 2009/61378, WO 2009/061379, WO 2009/061380, WO 2009/061381, WO 2009/098229, WO 2009/100102, WO 2010/115021, WO2010/115028, WO 2010/036515, WO 2011/082425, WO 2013/096305, WO 2013/184577, WO 2014/007921, WO 2014/164777, WO 2014/164800, WO 2014/164834, WO 2019/113413, WO 2019/113415, WO 2019/197318 (each of which is incorporated herein by reference).

Examples of suitable thermostable glucoamylases include, without limitation, the glucoamylases described in WO 2011/127802, WO 2013/036526, WO 2013/053801, WO 2018/164737, WO 2020/010101, and WO 2022/090564 (each of which is incorporated herein by reference).

Examples of suitable thermostable endoglucanases include, without limitation, the endoglucanases described in WO 2015/035914 (which is incorporated herein by reference) Examples of suitable thermostable lipases include, without limitation, the lipases described in WO 2017/112542 and WO 2020/014407 (which are both incorporated herein by reference).

Examples of suitable thermostable phytases include, without limitation, the phytases described in WO 1996/28567, WO 1997/33976, WO 1997/38096, WO 1997/48812, WO 1998/05785, WO 1998/06856, WO 1998/13480, WO 1998/20139, WO 1998/028408, WO 1999/48330, WO 1999/49022, WO 2003/066847, WO 2004/085638, WO 2006/037327, WO 2006/037328, WO 2006/038062, WO 2006/063588, WO 2007/112739, WO 2008/092901, WO 2008/116878, WO 2009/129489, and WO 2010/034835 (each of which is incorporated by reference). Commercially available phytase containing products include BIO-FEED PHYTASE™, PHYTASE NOVO™ CT or L, LIQMAX or RONOZYME™ NP, RONOZYME® HIPHOS, RONOZYME® P5000 (CT), NATUPHOS™ NG 5000.

Examples of suitable thermostable proteases include, without limitation, the proteases described in WO 1992/02614, WO 98/56926, WO 2001/151620, WO 2003/048353, WO 2006/086792, WO 2010/008841, WO 2011/076123, WO 2011/087836, WO 2012/088303, WO 2013/082486, WO 2014/209789, WO 2014/209800, WO 2018/098124, WO2018/118815 A1, and WO2018/169780A1 (each of which is incorporated herein by reference).

Suitable commercially available protease containing products include AVANTEC AMP®, FORTIVA REVO®, FORTIVA HEMI®.

Examples of suitable thermostable pullulanases include, without limitation, the pullulanases described in WO 2015/007639, WO 2015/110473, WO 2016/087327, WO 2017/014974, and WO 2020/187883 (each of which is incorporated herein by reference in its entirety). Suitable commercially available pullulanase products include PROMOZYME 400L, PROMOZYME™ D2 (Novozymes A/S, Denmark), OPTIMAX L-300 (Genencor Int., USA), and AMANO 8 (Amano, Japan).

Examples of suitable thermostable xylanases include, without limitation, the xylanases described in WO 2017/112540 and WO 2021/126966 (each of which is incorporated herein by reference). Suitable commercially available thermostable xylanase containing products include FORTIVA HEMI®.

The enzyme(s) described above are to be used in effective amounts in the processes of the present invention. Guidance for determining effective amounts of enzymes to be used in liquefying step (a) can be found in the published patent applications cited for each of the different thermostable liquefaction enzymes, along with guidance for performing activity assays for determining the activity of those enzymes.

### Saccharification Temperature

Saccharification may be performed at temperatures ranging from 20° C. to 75° C., from 30° C. to 70° C., or from 40° C. to 65° C. Preferably, the saccharification temperature is at least about 50° C., at least about 55° C., or at least about 60° C.

### Saccharification pH

Saccharification may occur at a ph ranging from 4 to 5. Preferably, the pH is about 4.5.

### Saccharification Time

Saccharification may last from about 24 hours to about 72 hours.

### Fermentation Time

Fermentation may last from 6 to 120 hours, from 24 hours to 96 hours, or from 35 hours to 60 hours.

### Simultaneous Saccharification and Fermentation

SSF may be performed at a temperature from 25° C. to 40° C., from 28° C. to 35° C., or from 30° C. to ° C., at a pH from 3.5 to 5 or from 3.8 to 4.3., for 24 to 96 hours, 36 to 72 hours, or from 48 to 60 hours. Preferably, SSF is performed at about 32° C., at a pH from 3.8 to 4.5 for from 48 to 60 hours.

Saccharification and/or Fermentation Enzymes

The present invention contemplates the use of enzymes during saccharifying step (b) and/or fermenting step (c). It is well known in the art to use various enzymes during saccharifying step (b) and/or fermenting step (c), including, for example, alpha-amylases, alpha-glucosidases, beta-amylases, beta-glucanases, beta-glucosidases, cellobiohydrolases, endoglucanases, glucoamylases, lipases, lytic polysaccharide monooxygenases (LPMOs), maltogenic alpha-amylases, pectinases, peroxidases, phytases, proteases, trehalases, and xylanases.

The enzymes used in saccharifying step (b) and/or fermenting step (c) may be added exogenously as mono-components or formulated as compositions comprising the enzymes. The enzymes used in saccharifying step (b) and/or fermenting step (c) may also be added via in situ expression from the fermenting organism (e.g., yeast).

Examples of suitable alpha-amylases include, without limitation, the alpha-amylases described in WO 2004/055178, WO 2006/069290, WO 2013/006756, WO 2013/034106, WO 2013/044867, WO 2021/163011, and WO 2021/163030 (each of which is incorporated herein by reference).

Examples of suitable glucoamylases include, without limitation, the glucoamylases described in WO 1984/02921, WO 1992/00381, WO 1999/28448, WO 2000/04136, WO 2001/04273, WO 2006/069289, WO 2011/066560, WO 2011/066576, WO 2011/068803, WO 2011/127802, WO 2012/064351, WO 2013/036526, WO 2013/053801, WO 2014/039773, WO 2014/177541, WO 2014/177546, WO 2016/062875, WO 2017/066255, and WO 2018/191215 (each of which is incorporated herein by reference.

Examples of suitable compositions comprising alpha-amylases and glucoamylases include, without limitation, the compostions described in WO 2006/069290, WO 2009/052101, WO 2011/068803, and WO 2013/006756 (each of which is incorporated by reference herein). Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL, SPIRIZYME ACHIEVE and AMG™ E (from Novozymes A/S); OPTIDEX™ 300, GC480, GC417 (from DuPont-Genencor); AMIGASE™ and AMIGASE™ PLUS (from DSM); G-ZYME™ G900, G-ZYME™ and G990 ZR (from DuPont-Genencor).

Examples of suitable beta-glucanases include, without limitation, the beta-glucanases described in WO 2021/055395 (which is incorporated herein by reference).

Examples of suitable beta-glucosidases include, without limitation, the beta-glucosidases described in WO 2005/047499, WO 2013/148993, WO 2014/085439 and WO 2012/044915 (each of which is incorporated herein by reference).

Examples of suitable cellobiohydrolases include, without limitation, the cellobiohydrolases described in WO 2013/148993, WO 2014/085439, WO 2014/138672, and WO 2016/040265 (each of which is incorporated herein by reference).

Examples of suitable endoglucanases include, without limitation, the endoglucanases described in WO 2013/148993 and WO 2014/085439 (both of which are incorporated herein by reference).

Examples of suitable maltogenic alpha-amylases are described in U.S. Pat. Nos. 4,598,048, 4,604,355 and 6,162,628, which are hereby incorporated by reference.

Examples of suitable lipases include, without limitation, the lipases described in WO 2017/112533, WO 2017/112539, and WO 2020/076697 (each of which is incorporated herein by reference).

Examples of suitable LPMOs include, without limitation, the LPMOs described in WO 2013/148993, WO 2014/085439, and WO 2019/083831 (each of which is incorporated herein by reference).

Examples of suitable phytases include, without limitation, the phytases described in WO 2001/62947 (which is incorporated herein by reference).

Examples of suitable pectinases include, without limitation, the pectinases described in WO 2022/173694 (which is incorporated herein by reference).

Examples of suitable peroxidases include, without limitation, the peroxidases described in WO 2019/231944 (which is incorporated herein by reference).

Examples of suitable proteases include, without limitation, the proteases described in WO 2017/050291, WO 2017/148389, WO 2018/015303, and WO 2018/015304 (each of which is incorporated herein by reference).

Examples of suitable trehalases include, without limitation, the trehalases described in WO 2016/205127, WO 2019/005755, WO 2019/030165, and WO 2020/023411 (each of which is incorporated herein by reference).

Examples of suitable xylanases include, without limitation, the xylanases described in WO 2016/005521, WO2019/055455, WO2020/160126, and WO 2021/026201 (each of which is incorporated herein by reference).

A commercially available xylanase-containing product is sold under the brand FIBEREX®.

Process for Producing a Fermentation Product from Ungelatinized Starch-Containing Material

An aspect of the invention relates to a process for reducing syrup viscosity at the backend of a process for producing a fermentation product (e.g., fuel ethanol) from an ungelatinized starch-containing material (i.e., granularized starch—often referred to as a “raw starch hydrolysis” process), wherein a laccase and/or a peroxidase is added after the fermenting step to decrease the quantity of particles in the thin stillage by increasing the size of particles in the whole stillage, thereby reducing the viscosity of syrup produced by evaporating the thin stillage.

In an embodiment, a process for producing a fermentation product from an ungelatinized starch-containing material comprises the following steps:
(a) saccharifying a starch-containing material at a temperature below the initial gelatinization temperature of the starch using a glucoamylase and an alpha-amylase to produce a fermentable sugar; and
(b) fermenting the sugar using a fermentation organism to produce a fermentation product;
(c) distilling the fermentation product to recover the fermentation product and produce whole stillage;
(d) processing the whole stillage to produce a thin stillage; and
(e) evaporating the thin stillage to produce a syrup;
wherein an oxidoreductase is added after the fermenting step to decrease the quantity of particles in the thin stillage by increasing the size of particles in the whole stillage, thereby reducing the viscosity of syrup produced by evaporating the syrup.

In an embodiment, the oxidoreductase is added to a beer well containing the beer prior to the distilling step. In an embodiment, the oxidoreductase is added during the distilling step. In an embodiment, the oxidoreductase is added to the whole stillage before the processing step.

In an embodiment, the processing step comprises a separation step performed by subjecting the whole stillage to a filtration centrifuge, a decanter centrifuge, a pressure screen, or a paddle screen. In an embodiment, the centrifuge is a decanter centrifuge, particularly a horizontal decanter centrifuge.

In an embodiment, the oxidoreductase is added to the whole stillage and incubated for a period of time before the whole stillage is subjected to the separation step.

In an embodiment, the oxidoreductase is a laccase. In an embodiment, the oxidoreductase is a peroxidase.

Preferably, the oxidoreductase used in the process has an optimum temperature that is compatible with the temperature ranges of the process step in which they are used. In an embodiment, the oxidoreductase is used at a temperature in the range from 30° C. to 100° C., such as 40° C. to 90° C., preferably 45° C. to 85° C.

Raw starch hydrolysis (RSH) processes are well-known in the art. The skilled artisan will appreciate that, except for the process parameters relating to liquefying step (a) which is not done in a RSH process, the process parameters described in Section II above are applicable to the process described in this section, including selection of the starch-containing material, reducing the grain particle size, saccharification temperature, time and pH, conditions for simultaneous saccharification and fermentation, and saccharification enzymes. The process parameters for an exemplary raw-starch hydrolysis process are described in further detail in WO 2004/106533 (which is incorporated herein by reference).

Examples of alpha-amylases that are preferably used in step (a) and/or step (b) include, without limitation, the alpha-amylases described in WO 2004/055178, WO 2005/003311, WO 2006/069290, WO 2013/006756, WO 2013/034106, WO 2021/163015, and WO 2021/163036 (each of which is incorporated by reference herein).

Examples of glucoamylases that are preferably used in step (a) and/or step (b) include, without limitation, WO 1999/28448, WO 2005/045018, WO2005/069840, WO 2006/069289 (each of which is incorporated by reference herein).

Examples of compositions comprising alpha-amylases and glucoamylase that are preferably used in step (a) and/or step (b) include, without limitation, the compositions described in WO 2015/031477 (which is incorporated by reference herein).

### Backend or Downstream Processing

### A. Recovery of the Fermentation Product and Production of Whole Stillage

Subsequent to fermentation or SSF, the fermentation product may be separated from the fermentation medium. The fermentation product, e.g., ethanol, can optionally be recovered from the fermentation medium using any method known in the art including, but not limited to, chromatography, electrophoretic procedures, differential solubility, distillation, or extraction. For example, alcohol is separated from the fermented starch-containing material and purified by conventional methods of distillation.

Thus, in one embodiment, the method of the invention further comprises distillation to obtain the fermentation product, e.g., ethanol. The fermentation and the distillation may be carried out simultaneously and/or separately/sequentially; optionally followed by one or more process steps for further refinement of the fermentation product. Following the completion of the distillation process, the material remaining is considered the whole stillage.

As another example, the desired fermentation product may be extracted from the fermentation medium by micro or membrane filtration techniques. Ethanol with a purity of up to about 96 vol. % can be obtained, which can be used as, for example, fuel ethanol, drinking ethanol, i.e., potable neutral spirits, or industrial ethanol.

In some embodiments of the methods, the fermentation product after being recovered is substantially pure. With respect to the methods herein, “substantially pure” intends a recovered preparation that contains no more than 15% impurity, wherein impurity intends compounds other than the fermentation product (e.g., ethanol). In one variation, a substantially pure preparation is provided wherein the preparation contains no more than 25% impurity, or no more than 20% impurity, or no more than 10% impurity, or no more than 5% impurity, or no more than 3% impurity, or no more than 1% impurity, or no more than 0.5% impurity.

Suitable assays to test for the production of ethanol and contaminants, and sugar consumption can be performed using methods known in the art. For example, ethanol product, as well as other organic compounds, can be analyzed by methods such as HPLC (High Performance Liquid Chromatography), GC-MS (Gas Chromatography Mass Spectroscopy) and LC-MS (Liquid Chromatography-Mass Spectroscopy) or other suitable analytical methods using routine procedures well known in the art. The release of ethanol in the fermentation broth can also be tested with the culture supernatant. Byproducts and residual sugar in the fermentation medium (e.g., glucose or xylose) can be quantified by HPLC using, for example, a refractive index detector for glucose and alcohols, and a UV detector for organic acids (Lin et al., Biotechnol. Bioeng. 90:775-779 (2005)), or using other suitable assay and detection methods well known in the art.

### B. Processing of Whole Stillage

In one embodiment, the whole stillage is processed into two streams-wet cake and centrate. The whole stillage is separated or partitioned into a solid and liquid phase by one or more methods for separating the centrate from the wet cake. The centrate is split into two flows—thin stillage, which goes to the evaporators, and backset, which is recycled to the front of the plant. Separating whole stillage into centrate (e.g., thin stillage when pumped toward the evaporators rather than the front end of the plant) and wet cake to remove a significant portion of the liquid/water, may be done using any suitable separation technique, including centrifugation, pressing and filtration. In a preferred embodiment, the separation/dewatering is carried out by centrifugation. Preferred centrifuges in industry are decanter type centrifuges, preferably high speed decanter type centrifuges. An example of a suitable centrifuge is the NX 400 steep cone series from ALFA LAVAL which is a high-performance decanter. A similar decanter centrifuge can also be purchased from FLOTTWEG. In another preferred embodiment, the separation is carried out using other conventional separation equipment such as a plate/frame filter presses, belt filter presses, screw presses, gravity thickeners and deckers, or similar equipment.

### C. Processing of Thin Stillage

Thin stillage is the term used for the supernatant of the centrifugation of the whole stillage. Typically, the thin stillage contains 4-8 percent dry solids (DS) (mainly proteins, soluble fiber, fats, fine fibers, and cell wall components) and has a temperature of about 60-90 degrees centigrade. The thin stillage stream may be condensed by evaporation to provide two process streams including: (i) an evaporator condensate stream comprising condensed water removed from the thin stillage during evaporation, and (ii) a syrup stream, comprising a more concentrated stream of the non-volatile dissolved and non-dissolved solids, such as non-fermentable sugars and oil, remaining present from the thin stillage as the result of removing the evaporated water.

Optionally, oil can be removed from the thin stillage or can be removed as an intermediate step to the evaporation process, which is typically carried out using a series of several evaporation stages.

Syrup and/or de-oiled syrup may be introduced into a dryer together with the wet cake (from the whole stillage separation step) to provide a product referred to as distillers dried grain with solubles, which also can be used as animal feed. In an embodiment, syrup and/or de-oiled syrup is sprayed into one or more dryers to combine the syrup and/or de-oiled syrup with the whole stillage to produce distillers dried grain with solubles.

Between 5-90 vol-%, such as between 10-80%, such as between 15-70%, such as between 20-60% of thin stillage (e.g., optionally hydrolyzed) may be recycled (as backset) to step (a). The recycled thin stillage (i.e., backset) may constitute from about 1-70 vol.-%, preferably 15-60% vol.-%, especially from about 30 to 50 vol.-% of the slurry formed in step (a). In an embodiment, the process further comprises recycling at least a portion of the thin stillage stream to the slurry, optionally after oil has been extracted from the thin stillage stream.

D. Drying of Wet Cake and Producing Distillers Dried Grains and Distillers Dried Grains with Solubles

After the wet cake, containing about 25-40 wt-%, preferably 30-38 wt-% dry solids, has been separated from the thin stillage (e.g., dewatered) it may be dried in a drum dryer, spray dryer, ring drier, fluid bed drier or the like in order to produce “Distillers Dried Grains” (DDG). DDG is a valuable feed ingredient for animals, such as livestock, poultry and fish. It is preferred to provide DDG with a content of less than about 10-12 wt.-% moisture to avoid mold and microbial breakdown and increase the shelf life. Further, high moisture content also makes it more expensive to transport DDG. The wet cake is preferably dried under conditions that do not denature proteins in the wet cake. The wet cake may be blended with syrup separated from the thin stillage and dried into DDG with Solubles (DDGS). Partially dried intermediate products, such as are sometimes referred to as modified wet distillers grains, may be produced by partially drying wet cake, optionally with the addition of syrup before, during or after the drying process.

### Exemplary Oxidoreductases Suitable for Use in the Processes of the Invention

Aspects of the invention relate to the addition of an oxidoreductase after the fermenting step of a process for producing a fermentation product to reduce syrup viscosity, for example, by decreasing the quantity of smaller sized particles in whole stillage so that the quantity of small particles that passes through the whole stillage processing step (e.g., decanting) into the thin stillage is decreased, thereby reducing the viscosity of the syrup produced by evaporating the thin stillage.

The present invention contemplates using any oxidoreductase that is capable of increasing the quantity of larger sized particles while simultaneously decreasing the quantity of smaller sized particles in whole stillage. Preferably, the oxidoreductase reduces the cumulative passing of particles having a size ranging from 100 μm to 1000 μm by at least 10 volume %, at least 15 volume %, at least 20 volume %, at least 25 volume %, at least 30 volume %, at least 35 volume %, at least 40 volume %, at least 45 volume %, at least 50 volume %, at least 55 volume %, at least 60 volume %, at least 65 volume %, at least 70 volume %, at least 75 volume %, or at least 80 volume %. Similarly, preferred oxidoreductases increase the distribution of particles in the whole stillage having a diameter greater than or equal to 400 μm by at least 15 volume %, at least 15 volume %, at least 20 volume %, at least 25 volume %, at least 30 volume %, at least 35 volume %, at least 40 volume %, at least 45 volume %, at least 50 volume %, at least 55 volume %%, at least 60 volume %, at least 65 volume %, at least 70 volume %, at least 75 volume %, or at least 80 volume %.

In an embodiment, the oxidoreductase is a laccase.

In an embodiment, the laccase is an AA1 laccase (EC 1.10.3.2).

A laccase suitable for use in a process of the invention may be obtained from the genus Thermothelomyces.

A laccase suitable for use in a process of the invention may be obtained from the species Thermothelomyces fergusii, Thermothelomyces guttulatus, Thermothelomyces heterothallicus, Thermothelomyces hinnuleus, Thermothelomyces myriococcoides, or Thermothelomyces thermophilus.

An exemplary laccase has the amino acid sequence of SEQ ID NO: 1. In an embodiment, the laccase has the amino acid sequence of SEQ ID NO: 1 with 0 to 10 conservative amino acid substitutions and has laccase activity. In an embodiment, the laccase is one with an amino acid sequence having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 1, and which has laccase activity.

A laccase suitable for use in a process of the invention may be obtained from the genus Coprinopsis.

A laccase suitable for use in a process of the invention may be obtained from the species Coprinus alcobae, Coprinus arachnoideus, Coprinus asterophoroides, Coprinus cinerea, Coprinus clastophyllus, Coprinus colosseus, Coprinus comatus, Coprinus coniophorus, Coprinus cordisporus, Coprinus cortinatus, Coprinus ephemerus, Coprinus fissolanatus, Coprinus foetidellus, Coprinus goudensis, Coprinus latisporus, Coprinus littoralis, Coprinus maysoidisporus, Coprinus myceliocephalus, Coprinus palmeranus, Coprinus patouillardii, Coprinus phaeopunctatus, Coprinus pinetorum, Coprinus aff radians PP63, Coprinus roseistipitatus, Coprinus rufopruinatus, Coprinus simulans, Coprinus spadiceisporus, Coprinus sterquilinus, Coprinus subdomesticus, Coprinus trigonosporus, Coprinus vosoustii, or Coprinus xerophilus.

An exemplary laccase has the amino acid sequence of SEQ ID NO: 2. In an embodiment, the laccase has the amino acid sequence of SEQ ID NO: 2 with 0 to 10 conservative amino acid substitutions and has laccase activity. In an embodiment, the laccase is one with an amino acid sequence having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 2, and which has laccase activity.

A laccase suitable for use in a process of the invention may be obtained from the genus Polyporus.

A laccase suitable for use in a process of the invention may be obtained from the species Polyporus americanus, Polyporus anthracophilus, Polyporus arcularioides, Polyporus arcularius, Polyporus auratus, Polyporus austrosinensis, Polyporus brasiliensis, Polyporus brevibasidiosus, Polyporus brumalis, Polyporus chozeniae, Polyporus ciliates, Polyporus corylinus, Polyporus cryptopus, Polyporus curtipes, Polyporus cuticulatus, Polyporus decurrens, Polyporus dictyopus, Polyporus elongoporus, Polyporus foedatus, Polyporus fraxineus, Polyporus frondosus, Polyporus gayanus, Polyporus grammocephalus, Polyporus guianensis, Polyporus hapalopus, Polyporus hartmannii, Polyporus hemicapnodes, Polyporus hypomelanus, Polyporus koreanus, Polyporus lamelliporus, Polyporus lepideus, Polyporus leprieurii, Polyporus leptocephalus, Polyporus longiporus, Polyporus mangshanensis, Polyporus marianiae, Polyporus mcmurphyi, Polyporus melanopus, Polyporus meridionalis, Polyporus minutosquamosus, Polyporus orientivarius, Polyporus parvovarius, Polyporus philippinensis, Polyporus pinsitus, Polyporus plorans, Polyporus pseudobetulinus, Polyporus radicatus, Polyporus roseofuscus, Polyporus sagranus, Polyporus squamulosus, Polyporus subvarius, Polyporus tessellatus, Polyporus thailandensis, Polyporus tricholoma, Polyporus tsugae, Polyporus tuberaster, Polyporus tucumanensis, Polyporus tumulosus, Polyporus ulleungus, Polyporus umbellatus, or Polyporus varius.

An exemplary laccase has the amino acid sequence of SEQ ID NO: 3. In an embodiment, the laccase has the amino acid sequence of SEQ ID NO: 3 with 0 to 10 conservative amino acid substitutions and has laccase activity. In an embodiment, the laccase is one with an amino acid sequence having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 3, and which has laccase activity.

In an embodiment, the oxidoreductase is a peroxidase.

In an embodiment, the peroxidase is an AA2 peroxidase (EC 1.11.1.7).

A peroxidase suitable for use in a process of the invention may be obtained from the genus Coprinus.

A peroxidase suitable for use in a process of the invention may be obtained from the species Coprinus alcobae, Coprinus arachnoideus, Coprinus asterophoroides, Coprinus cinerea, Coprinus clastophyllus, Coprinus colosseus, Coprinus comatus, Coprinus coniophorus, Coprinus cordisporus, Coprinus cortinatus, Coprinus ephemerus, Coprinus fissolanatus, Coprinus foetidellus, Coprinus goudensis, Coprinus latisporus, Coprinus littoralis, Coprinus maysoidisporus, Coprinus myceliocephalus, Coprinus palmeranus, Coprinus patouillardii, Coprinus phaeopunctatus, Coprinus pinetorum, Coprinus aff radians PP63, Coprinus roseistipitatus, Coprinus rufopruinatus, Coprinus simulans, Coprinus spadiceisporus, Coprinus sterquilinus, Coprinus subdomesticus, Coprinus trigonosporus, Coprinus vosoustii, or Coprinus xerophilus.

An exemplary peroxidase has the amino acid sequence of SEQ ID NO: 4. In an embodiment, the peroxidase has the amino acid sequence of SEQ ID NO: 4 with 0 to 10 conservative amino acid substitutions and has peroxidase activity. In an embodiment, the peroxidase is one with an amino acid sequence having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 4, and which has peroxidase activity.

A peroxidase suitable for use in a process of the invention may be obtained from the genus Glycine.

A peroxidase suitable for use in a process of the invention may be obtained from the species Glycine gracilis, Glycine max, or Glycine soja.

An exemplary peroxidase has the amino acid sequence of SEQ ID NO: 5. In an embodiment, the peroxidase has the amino acid sequence of SEQ ID NO: 5 with 0 to 10 conservative amino acid substitutions and has peroxidase activity. In an embodiment, the peroxidase is one with an amino acid sequence having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 5, and which has peroxidase activity.

A peroxidase suitable for use in a process of the invention may be obtained from the genus Armoracia.

A peroxidase suitable for use in a process of the invention may be obtained from the species Armoracia rusticana.

An exemplary peroxidase has the amino acid sequence of SEQ ID NO: 6. In an embodiment, the peroxidase has the amino acid sequence of SEQ ID NO: 6 with 0 to 10 conservative amino acid substitutions and has peroxidase activity. In an embodiment, the peroxidase is one with an amino acid sequence having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 6, and which has peroxidase activity.

Preferably, the oxidoreductase has a temperature optimum ranging from 30° C. to 100° C., such as 40° C. to 90° C., preferably 45° C. to 85° C.

The oxidoreductases may be dosed in the beer, beer well, distillation, and/or whole stillage in a concentration of between 0.0001-1 mg EP (Enzyme Protein)/g DS, e.g., 0.0005-0.5 mg EP/g DS, such as 0.001-0.1 mg EP/g DS.

### Exemplary Fermenting Organisms

Aspects of the invention relate to the use of a fermenting organism for producing a fermentation product. Especially suitable fermenting organisms are able to ferment, i.e., convert, sugars, such as arabinose, glucose, maltose and/or xylose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Examples of commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties). Other useful yeast strains are available from biological depositories such as the American Type Culture Collection (ATCC) or the Deutsche Sammlung von Mikroorganismen und Zellkulturen GmbH (DSMZ), such as, e.g., BY4741 (e.g., ATCC 201388); Y108-1 (ATCC PTA.10567) and NRRL YB-1952 (ARS Culture Collection). Still other S. cerevisiae strains suitable as host cells DBY746, [Alpha][Eta]22, S150-2B, GPY55-15Ba, CEN.PK, USM21, TMB3500, TMB3400, VTT-A-63015, VTT-A-85068, VTT-c-79093 and their derivatives as well as Saccharomyces sp. 1400, 424A (LNH-ST), 259A (LNH-ST) and derivatives thereof.

As used herein, a “derivative” of strain is derived from a referenced strain, such as through mutagenesis, recombinant DNA technology, mating, cell fusion, or cytoduction between yeast strains. Those skilled in the art will understand that the genetic alterations, including metabolic modifications exemplified herein, may be described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art can apply the teachings and guidance provided herein to other organisms. For example, the metabolic alterations exemplified herein can readily be applied to other species by incorporating the same or analogous encoding nucleic acid from species other than the referenced species.

The fermenting organism may be Saccharomyces strain, e.g., Saccharomyces cerevisiae strain produced using the method described and concerned in U.S. Pat. No. 8,257,959-BB. In one embodiment, the recombinant cell is a derivative of a strain Saccharomyces cerevisiae CIBTS1260 (deposited under Accession No. NRRL Y-50973 at the Agricultural Research Service Culture Collection (NRRL), Illinois 61604 U.S.A.).

The fermenting organism may also be a derivative of Saccharomyces cerevisiae strain NMI V14/004037 (See, WO2015/143324 and WO2015/143317 each incorporated herein by reference), strain nos. V15/004035, V15/004036, and V15/004037 (See, WO 2016/153924 incorporated herein by reference), strain nos. V15/001459, V15/001460, V15/001461 (See, WO2016/138437 incorporated herein by reference), strain no. NRRL Y67342 (See, WO2018/098381 incorporated herein by reference), strain nos. NRRL Y67549 and NRRL Y67700 (See, WO 2019/161227 incorporated herein by reference), or any strain described in WO2017/087330 (incorporated herein by reference).

The fermenting organisms may comprise one or more heterologous polynucleotides encoding an alpha-amylase, glucoamylase, protease and/or cellulase. Examples of alpha-amylase, glucoamylase, protease and cellulases suitable for expression in the fermenting organism are known in the art (See, WO2021/231623 incorporated herein by reference),

The fermenting organism may be in the form of a composition comprising a fermenting organism and a naturally occurring and/or a non-naturally occurring component. The fermenting organism may be in any viable form, including crumbled, dry, including active dry and instant, compressed, cream (liquid) form etc. In one embodiment, the fermenting organism (e.g., a Saccharomyces cerevisiae yeast strain) is dry yeast, such as active dry yeast or instant yeast. In one embodiment, the fermenting organism is crumbled yeast. In one embodiment, the fermenting organism is a compressed yeast. In one embodiment, the fermenting organism is cream yeast.

In one embodiment is a composition comprising a fermenting organism described herein (e.g., a Saccharomyces cerevisiae yeast strain), and one or more of the components selected from the group consisting of: surfactants, emulsifiers, gums, swelling agent, and antioxidants and other processing aids.

The compositions described herein may comprise a fermenting organism described herein (e.g., a Saccharomyces cerevisiae yeast strain) and any suitable surfactants. In one embodiment, the surfactant(s) is/are an anionic surfactant, cationic surfactant, and/or nonionic surfactant.

The compositions described herein may comprise a fermenting organism described herein (e.g., a Saccharomyces cerevisiae yeast strain) and any suitable emulsifier. In one embodiment, the emulsifier is a fatty-acid ester of sorbitan. In one embodiment, the emulsifier is selected from the group of sorbitan monostearate (SMS), citric acid esters of monodiglycerides, polyglycerolester, fatty acid esters of propylene glycol.

In one embodiment, the composition comprises a fermenting organism described herein (e.g., a Saccharomyces cerevisiae yeast strain), and Olindronal SMS, Olindronal SK, or Olindronal SPL including composition concerned in European Patent No. 1,724,336 (hereby incorporated by reference). These products are commercially available from Bussetti, Austria, for active dry yeast.

The compositions described herein may comprise a fermenting organism described herein (e.g., a Saccharomyces cerevisiae yeast strain) and any suitable gum. In one embodiment, the gum is selected from the group of carob, guar, tragacanth, arabic, xanthan and acacia gum, in particular for cream, compressed and dry yeast.

The compositions described herein may comprise a fermenting organism described herein (e.g., a Saccharomyces cerevisiae yeast strain) and any suitable swelling agent. In one embodiment, the swelling agent is methyl cellulose or carboxymethyl cellulose.

The compositions described herein may comprise a fermenting organism described herein (e.g., a Saccharomyces cerevisiae yeast strain) and any suitable anti-oxidant. In one embodiment, the antioxidant is butylated hydroxyanisol (BHA) and/or butylated hydroxytoluene (BHT), or ascorbic acid (vitamin C), particular for active dry yeast.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 105 to 10¹², preferably from 10⁷ to 10¹⁰, especially about 5×10⁷.

The invention is further summarized in the following paragraphs:

### What is Claimed is:

1. A process for reducing the viscosity of syrup, the process comprising:
(a) liquefying a starch-containing material with a thermostable alpha-amylase at a temperature above the initial gelatinization temperature of the starch to produce a dextrin;
(b) saccharifying the dextrin with a glucoamylase to produce a fermentable sugar;
(c) fermenting the sugar with a fermenting organism to produce a beer comprising the fermentation product;
(d) distilling the beer to recover the fermentation product and produce a whole stillage;
(e) processing the whole stillage to produce a thin stillage; and
(f) evaporating the thin stillage to produce a syrup;
wherein an oxidoreductase is added after the fermenting step to decrease the quantity of particles in the thin stillage by increasing the size of particles in the whole stillage, thereby reducing the viscosity of syrup produced by evaporating the thin stillage.
2. A process for reducing viscosity of syrup in a producing a fermentation product from a starch-containing material, the process comprising:
(a) saccharifying a starch-containing material at a temperature below the initial gelatinization temperature of the starch with an alpha-amylase and a glucoamylase to produce fermentable a sugar;
(b) fermenting the sugar with a fermenting organism to produce a beer comprising the fermentation product;
(c) distilling the beer to recover the fermentation product and to produce a whole stillage;
(d) processing the whole stillage to produce a thin stillage; and
(e) evaporating the thin stillage to produce a syrup;
wherein an oxidoreductase is added after the fermenting step to decrease the quantity of particles in the thin stillage by increasing the size of particles in the whole stillage, thereby reducing the viscosity of syrup produced by evaporating the syrup.
3. A process for reducing the viscosity of syrup, the process comprising:
(a) liquefying a starch-containing material with a thermostable alpha-amylase at a temperature above the initial gelatinization temperature of the starch to produce a dextrin;
(b) saccharifying the dextrin with a glucoamylase to produce a fermentable sugar;
(c) fermenting the sugar with a fermenting organism to produce a beer comprising the fermentation product;
(d) distilling the beer to recover the fermentation product and produce a whole stillage;
(e) processing the whole stillage to produce a thin stillage; and
(f) evaporating the thin stillage to produce a syrup;
wherein an oxidoreductase is added to the beer after the fermenting step and before the distilling step to decrease the quantity of particles in the thin stillage by increasing the size of particles in the whole stillage, thereby reducing the viscosity of syrup produced by evaporating the thin stillage.
4. A process for reducing viscosity of syrup in a producing a fermentation product from a starch-containing material, the process comprising:
(a) saccharifying a starch-containing material at a temperature below the initial gelatinization temperature of the starch with an alpha-amylase and a glucoamylase to produce fermentable a sugar;
(b) fermenting the sugar with a fermenting organism to produce a beer comprising the fermentation product;
(c) distilling the beer to recover the fermentation product and to produce a whole stillage;
(d) processing the whole stillage to produce a thin stillage; and
(e) evaporating the thin stillage to produce a syrup;
wherein an oxidoreductase is added to the beer after the fermenting step and before the distilling step to decrease the quantity of particles in the thin stillage by increasing the size of particles in the whole stillage, thereby reducing the viscosity of syrup produced by evaporating the syrup.
5. A process for reducing the viscosity of syrup, the process comprising:
(a) liquefying a starch-containing material with a thermostable alpha-amylase at a temperature above the initial gelatinization temperature of the starch to produce a dextrin;
(b) saccharifying the dextrin with a glucoamylase to produce a fermentable sugar;
(c) fermenting the sugar with a fermenting organism to produce a beer comprising the fermentation product;
(d) distilling the beer to recover the fermentation product and produce a whole stillage;
(e) processing the whole stillage to produce a thin stillage; and
(f) evaporating the thin stillage to produce a syrup;
wherein an oxidoreductase is added after the fermenting step during the distilling step and before the processing step to decrease the quantity of particles in the thin stillage by increasing the size of particles in the whole stillage, thereby reducing the viscosity of syrup produced by evaporating the thin stillage.
6. A process for reducing viscosity of syrup in a producing a fermentation product from a starch-containing material, the process comprising:
(a) saccharifying a starch-containing material at a temperature below the initial gelatinization temperature of the starch with an alpha-amylase and a glucoamylase to produce fermentable a sugar;
(b) fermenting the sugar with a fermenting organism to produce a beer comprising the fermentation product;
(c) distilling the beer to recover the fermentation product and to produce a whole stillage;
(d) processing the whole stillage to produce a thin stillage; and
(e) evaporating the thin stillage to produce a syrup;
wherein an oxidoreductase is added after the fermenting step during the distilling step and before the processing step to decrease the quantity of particles in the thin stillage by increasing the size of particles in the whole stillage, thereby reducing the viscosity of syrup produced by evaporating the syrup.
7. A process for reducing the viscosity of syrup, the process comprising:
(a) liquefying a starch-containing material with a thermostable alpha-amylase at a temperature above the initial gelatinization temperature of the starch to produce a dextrin;
(b) saccharifying the dextrin with a glucoamylase to produce a fermentable sugar;
(c) fermenting the sugar with a fermenting organism to produce a beer comprising the fermentation product;
(d) distilling the beer to recover the fermentation product and produce a whole stillage;
(e) processing the whole stillage to produce a thin stillage; and
(f) evaporating the thin stillage to produce a syrup;
wherein an oxidoreductase is added to the whole stillage after the distilling step and before the processing to decrease the quantity of particles in the thin stillage by increasing the size of particles in the whole stillage, thereby reducing the viscosity of syrup produced by evaporating the thin stillage.
8. A process for reducing viscosity of syrup in a producing a fermentation product from a starch-containing material, the process comprising:
(a) saccharifying a starch-containing material at a temperature below the initial gelatinization temperature of the starch with an alpha-amylase and a glucoamylase to produce fermentable a sugar;
(b) fermenting the sugar with a fermenting organism to produce a beer comprising the fermentation product;
(c) distilling the beer to recover the fermentation product and to produce a whole stillage;
(d) processing the whole stillage to produce a thin stillage; and
(e) evaporating the thin stillage to produce a syrup;
wherein an oxidoreductase is added to the whole stillage after the distilling step and before the processing to decrease the quantity of particles in the thin stillage by increasing the size of particles in the whole stillage, thereby reducing the viscosity of syrup produced by evaporating the syrup.
9. The process of any one of paragraphs 1 to 8, wherein the processing step comprises a separation step performed by subjecting the whole stillage to a filtration centrifuge, a decanter centrifuge, a pressure screen, or a paddle screen.
10. The process of paragraph 9, wherein the centrifuge is a decanter centrifuge, particularly a horizontal decanter centrifuge.
11. The process of any one of paragraphs 1 to 10, wherein the laccase and/or the peroxidase is added to the whole stillage and incubated for a period of time before the whole stillage is subjected to the separation step.
12. The process of any one of paragraphs 1 to 11, wherein the laccase and/or peroxidase is used at a temperature in the range from 30° C. to 100° C., such as 40° C. to 90° C., preferably 45° C. to 85° C.
13. The process of any one of paragraphs 1 to 12, wherein the cumulative passing is reduced by from 10 volume % to 80 volume % for particles having a size ranging from 100 μm to 1000 μm.
14. The process of any one of paragraphs 1 to 13, wherein the cumulative passing of particles having a diameter of 400 μm is decreased by 10 volume % to 80 volume %.
15. The process of any one of paragraphs 1 to 14, wherein the oxidoreductase is a laccase.
16. The process of paragraph 15, wherein the laccase is an AA1 laccase (EC 1.10.3.2).
17. The process of any one of paragraphs 15 to 16, wherein the laccase is from the genus Thermothelomyces.
18. The process of any one of paragraphs 15 to 17, wherein the laccase is from the species Thermothelomyces fergusii, Thermothelomyces guttulatus, Thermothelomyces heterothallicus, Thermothelomyces hinnuleus, Thermothelomyces myriococcoides, or Thermothelomyces thermophilus.
19. The process of any one of paragraphs 15 to 18, wherein the laccase has the amino acid sequence of SEQ ID NO: 1 with from 0 to 10 conservative amino acid substitutions, or is one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 1, and which has laccase activity.
20. The process of paragraph 15, wherein the laccase is from the genus Coprinopsis.
21. The process of paragraph 15 or 20, wherein the laccase is from the species Coprinus alcobae, Coprinus arachnoideus, Coprinus asterophoroides, Coprinus cinerea, Coprinus clastophyllus, Coprinus colosseus, Coprinus comatus, Coprinus coniophorus, Coprinus cordisporus, Coprinus cortinatus, Coprinus ephemerus, Coprinus fissolanatus, Coprinus foetidellus, Coprinus goudensis, Coprinus latisporus, Coprinus littoralis, Coprinus maysoidisporus, Coprinus myceliocephalus, Coprinus palmeranus, Coprinus patouillardii, Coprinus phaeopunctatus, Coprinus pinetorum, Coprinus aff radians PP63, Coprinus roseistipitatus, Coprinus rufopruinatus, Coprinus simulans, Coprinus spadiceisporus, Coprinus sterquilinus, Coprinus subdomesticus, Coprinus trigonosporus, Coprinus vosoustii, or Coprinus xerophilus.
22. The process of any one of paragraphs 15 or 21 to 22, wherein the laccase has the amino acid sequence of SEQ ID NO: 2 with from 0 to 10 conservative amino acid substitutions, or is one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 2, and which has laccase activity.
23. The process of paragraph 15, wherein the laccase is from the genus Polyporus.
24. The process of paragraph 15 or 23, wherein the laccase is from the species Polyporus americanus, Polyporus anthracophilus, Polyporus arcularioides, Polyporus arcularius, Polyporus auratus, Polyporus austrosinensis, Polyporus brasiliensis, Polyporus brevibasidiosus, Polyporus brumalis, Polyporus chozeniae, Polyporus ciliates, Polyporus corylinus, Polyporus cryptopus, Polyporus curtipes, Polyporus cuticulatus, Polyporus decurrens, Polyporus dictyopus, Polyporus elongoporus, Polyporus foedatus, Polyporus fraxineus, Polyporus frondosus, Polyporus gayanus, Polyporus grammocephalus, Polyporus guianensis, Polyporus hapalopus, Polyporus hartmannii, Polyporus hemicapnodes, Polyporus hypomelanus, Polyporus koreanus, Polyporus lamelliporus, Polyporus lepideus, Polyporus leprieurii, Polyporus leptocephalus, Polyporus longiporus, Polyporus mangshanensis, Polyporus marianiae, Polyporus mcmurphyi, Polyporus melanopus, Polyporus meridionalis, Polyporus minutosquamosus, Polyporus orientivarius, Polyporus parvovarius, Polyporus philippinensis, Polyporus pinsitus, Polyporus plorans, Polyporus pseudobetulinus, Polyporus radicatus, Polyporus roseofuscus, Polyporus sagranus, Polyporus squamulosus, Polyporus subvarius, Polyporus tessellatus, Polyporus thailandensis, Polyporus tricholoma, Polyporus tsugae, Polyporus tuberaster, Polyporus tucumanensis, Polyporus tumulosus, Polyporus ulleungus, Polyporus umbellatus, or Polyporus varius.
25. The process of any one of paragraphs 15 or 23 to 24, wherein the laccase has the amino acid sequence of SEQ ID NO: 3 with from 0 to 10 conservative amino acid substitutions, or is one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 3, and which has laccase activity.
26. The process of any one of paragraphs 1 to 14, wherein the oxidoreductase is a peroxidase.
27. The process of paragraph 26, wherein the peroxidase is an AA2 peroxidase (EC 1.11.1.7).
28. The process of paragraphs 26 or 27, wherein the peroxidase is from the genus Coprinus.
29. The process of any one of paragraphs 26 to 28, wherein the peroxidase is from the species Coprinus alcobae, Coprinus arachnoideus, Coprinus asterophoroides, Coprinus cinerea, Coprinus clastophyllus, Coprinus colosseus, Coprinus comatus, Coprinus coniophorus, Coprinus cordisporus, Coprinus cortinatus, Coprinus ephemerus, Coprinus fissolanatus, Coprinus foetidellus, Coprinus goudensis, Coprinus latisporus, Coprinus littoralis, Coprinus maysoidisporus, Coprinus myceliocephalus, Coprinus palmeranus, Coprinus patouillardii, Coprinus phaeopunctatus, Coprinus pinetorum, Coprinus aff radians PP63, Coprinus roseistipitatus, Coprinus rufopruinatus, Coprinus simulans, Coprinus spadiceisporus, Coprinus sterquilinus, Coprinus subdomesticus, Coprinus trigonosporus, Coprinus vosoustii, or Coprinus xerophilus.
30. The process of any one of paragraphs 26 to 29, wherein the peroxidase has the amino acid sequence of SEQ ID NO: 4 with from 0 to 10 conservative amino acid substitutions, or is one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 4, and which has peroxidase activity.
31. The process of paragraph 26, wherein the peroxidase is from the genus Glycine.
32. The process of paragraph 31, wherein the peroxidase is from the species Glycine gracilis, Glycine max, or Glycine soja.
33. The process of paragraph 31 or 32, wherein the peroxidase has the amino acid sequence of SEQ ID NO: 5 with from 0 to 10 conservative amino acid substitutions, or is one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 5, and which has peroxidase activity.
34. The process of paragraph 26, wherein the peroxidase is from the genus Armoracia.
35. The process of paragraph 31, wherein the peroxidase is from the species 20 Armoracia rusticana.
36. The process of paragraph 34 or 35, wherein the peroxidase has the amino acid sequence of SEQ ID NO: 6 with from 0 to 10 conservative amino acid substitutions, or is one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 6, and which has peroxidase activity.
37. The process of any one of claims 1 to 36, wherein the saccharifying and fermenting steps are performed simultaneously.
38. The process of any one of claims 1 to 37, wherein the starch-containing material comprises beets, maize, corn, wheat, rye, barley, oats, triticale, sorghum, sweet potatoes, rice, millet, pearl millet, and/or foxtail millet.
39. The process of any one of claims 1 to 38, wherein the fermentation product is ethanol, preferably fuel ethanol.
40. The process of any one of claims 1 to 39, wherein the fermenting organism is yeast.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. Various references are cited herein, the disclosures of which are incorporated herein by reference in their entireties. The present invention is further described by the following examples which should not be construed as limiting the scope of the invention.

### Materials & Methods

### Enzymes Used in the Examples

Oxidoreductase A: exemplary laccase from Thermothelomyces thermophilus disclosed in SEQ ID NO: 1
Oxidoreductase B: exemplary peroxidase from Coprinopsis cinerea disclosed in SEQ ID NO: 4

### Determination of Td by Differential Scanning Calorimetry for Liquefaction Enzymes

The thermostability of an enzyme is determined by Differential Scanning Calorimetry (DSC) using a VP-Capillary Differential Scanning Calorimeter (MICROCAL Inc., Piscataway, NJ, USA). The thermal denaturation temperature, Td (° C.), is taken as the top of denaturation peak (major endothermic peak) in thermograms (Cp vs. T) obtained after heating enzyme solutions (approx. 0.5 mg/ml) in buffer (50 mM acetate, pH 5.0) at a constant programmed heating rate of 200 K/hr.

Sample- and reference-solutions (approx. 0.2 ml) are loaded into the calorimeter (reference: buffer without enzyme) from storage conditions at 10° C. and thermally pre-equilibrated for 20 minutes at 20° C. prior to DSC scan from 20° C. to 120° C. Denaturation temperatures are determined at an accuracy of approximately +/−1° C.

### EXAMPLES

### Example 1: Characterizing Solids in Thin Stillage and Whole Stillage from a Commercial Ethanol Plant

Whole stillage and thin stillage were obtained from a commercial ethanol plant in the midwest USA. In particle size distribution testing, about 4.5 ml of well mixed stillage was diluted with 20 ml DI water. The diluted stillage was analyzed using Ls13 320 Laser Diffraction particle Size Analyzer (BECKMAN COULTER), particle size measurements are in duplicate. In solid content measurement, a 9 ml thin stillage was mixed with 20 ml deionized water in 50 ml tube. After centrifuging at 5300 rpm for 10 minutes using a floor centrifuge AVANTI J-E (BECKMAN COULTER), supernatant was removed. The solid was frozen in −20 C freezer and then dried in AssetRD-184 freezer drier (LABCONCO). Solid content measurements are in triplicate. The % solid content was obtained by dividing dry solid by weight of 9 ml thin stillage.

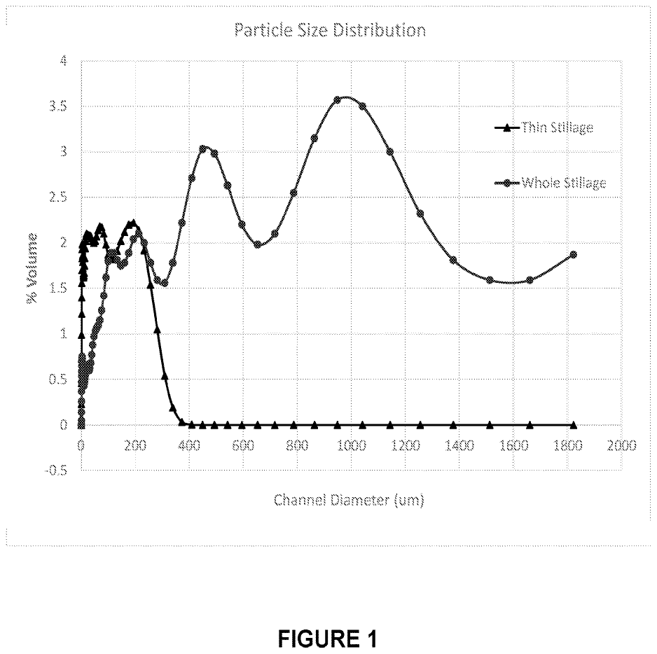

FIG. 1 shows that the particle diameter in thin stillage is under 400 μm, while whole stillage has particle diameter in a range of 0 to 1820 μm. The data indicated that decanting in this ethanol production plant has kept particles having a diameter above 400 μm in wet cake. Although the cut off particle diameter may vary from plant to plant due to equipment setting, it should not differ significantly from 400 μm among ethanol plants. The solid content of thin stillage is averaged +/−standard deviation at 3.91%+/−0.15%.

### Example 2: Increase Particle Size by Treating Whole Stillage with Oxidoreductase at 65° C.

The whole stillage used in this example was obtained from a commercial ethanol plant in the midwest USA. The whole stillage has 12.25% average % dry solid measured using moisture analyzer and a pH of about 5.1. About 10 g whole stillage was weighed in each tube. Oxidoreductase A and Oxidoreductase B were used in this experiment. Hydrogen peroxide was diluted to 0.06% and added at 100 ul/tube based on Table 1 below. All samples were vortexed every 10 minutes during their incubation in 65° C. water bath. After 60 minutes of treatment, all samples were cooled down and stored in refrigerator prior to particle size distribution testing.

TABLE 1
Treatment of whole stillage with laccase and peroxidase at 65° C. for 1 hour

| | Whole | Oxidoreductase | Oxidoreductase B | 0.06% | Temp. | Time |
|---|---|---|---|---|---|---|
| Tube | Stillage g | A (μg/gDS) | (μg/gDS) | H₂O₂ (μl) | ° C. | (hour) |
| 1 | 9.615 | | | | 65 | 1 |
| 2 | 9.613 | 10 | | | 65 | 1 |
| 3 | 9.616 | 40 | | | 65 | 1 |
| 4 | 9.538 | | 10 | 100 | 65 | 1 |
| 5 | 9.541 | | 40 | 100 | 65 | 1 |
| 6 | 9.773 | | 10 | | 65 | 1 |
| 7 | 9.564 | | 40 | | 65 | 1 |

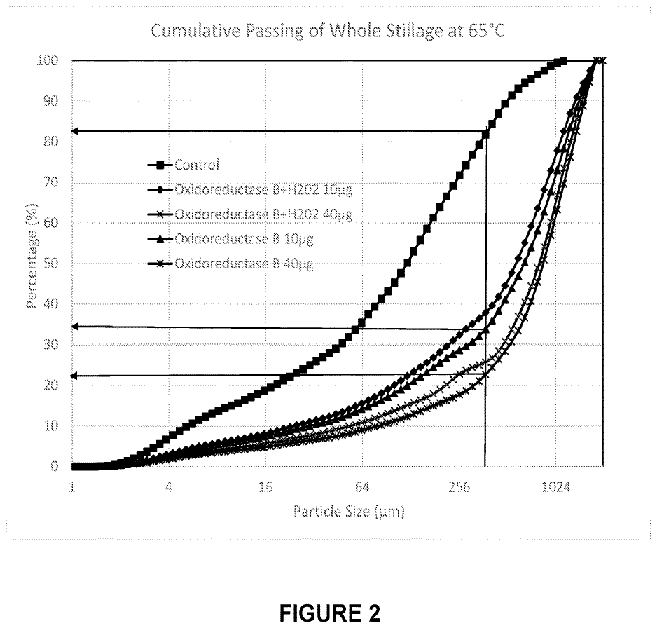

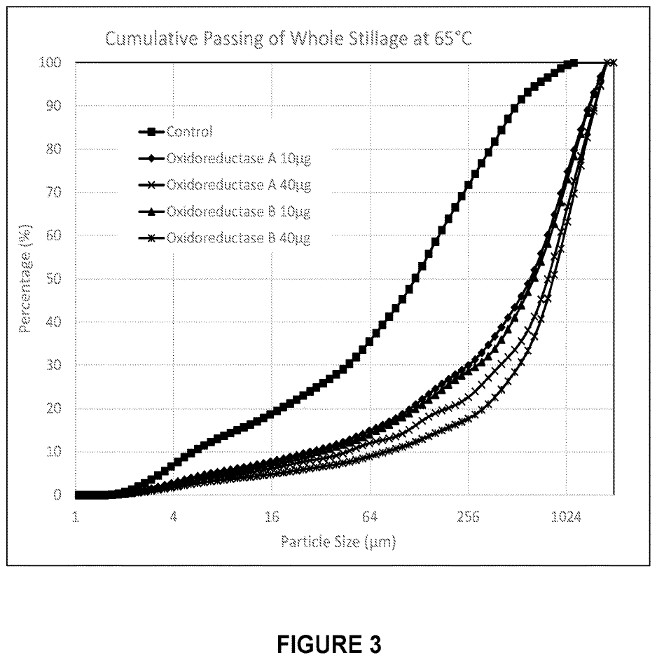

The treated whole stillage (WS) samples were diluted by adding 2 ml slurry into 10 ml deionized water. Each diluted sample was analyzed using Ls13 320 Laser Diffraction particle Size Analyzer (BECKMAN COULTER), average of WS particle size measurements was used in the figures. FIG. 2 and FIG. 3 show the increased WS particle size resulting from treatment with the oxidoreductases. In each figure, percentage (y-axis) is the cumulative volume % of all particles that are less than or equal to a specific particle size (x-axis) of WS. At a particle size of 400 μm diameter, 10 and 40 μg/g dry solid (DS) of Oxidoreductase A treatment reduces the cumulative passing of particles from 81 volume % (no oxidoreductase control) to 35 volume % and 22 volume %, as illustrated in FIG. 2. A similar trend of a reduction in the cumulative passing of particles having a size of 400 μm diameter in whole stillage can be observed for Oxidoreductase B treatment, as illustrated in FIG. 3.

The impact of particle diameter by oxidoreductase treatment is shown in Table 2 below. The particles sized at or less than a specific diameter (μm) are shown at various levels of particle volume percentage. At 50% particle volume, no enzyme control and 10 μg Oxidoreductase A treated whole stillages have particle size s 133.1 μm and ≤676.5 μm, respectively, indicating more small particles in the no enzyme control sample than the 10 μg Oxidoreductase A treated sample. In other words, 10 μg Oxidoreductase A treatment reduced the quantity of smaller sized particles and increased the quantity of larger sized particles. Similarly, Oxidoreductase B treatment with or without peroxide also decreased the volume % of smaller sized particles and increased the volume % of larger sized particles in the whole stillage.

TABLE 2
Impact of particle diameter by laccase and peroxidase treatment at 65° C.

| | Particle Diameter (≤ um) | | | | | | |
|---|---|---|---|---|---|---|---|
| | No | | | | | | |
| Volume | enzyme | Laccase | Laccase | Peroxidase | Peroxidase | Peroxidase | Peroxidase |
| % | Control | 10 μg | 40 μg | 10 ug + H₂O₂ | 40 ug + H₂O₂ | 10 μg | 40 μg |
| 10 | 5.8 | 30.2 | 50.6 | 26.9 | 59.2 | 33.2 | 87.7 |
| 25 | 33.4 | 183.6 | 329.9 | 168.7 | 375.0 | 204.4 | 463.0 |
| 50 | 133.1 | 676.5 | 865.1 | 633.4 | 881.8 | 711.6 | 933.8 |
| 75 | 319.0 | 1148.0 | 1306.0 | 1085.0 | 1285.0 | 1184.0 | 1355.0 |
| 90 | 557.7 | 1549.0 | 1674.0 | 1480.0 | 1633.0 | 1567.0 | 1693.0 |

### Example 3: Increase Particle Size by Treating Whole Stillage with Oxidoreductases at 50° C.

The whole stillage from Example 2 was used in this example at 10 g/tube. Oxidoreductase A and Oxidoreductase B from example 2 were used and dosed at 10 and 100 μg/gDS. A 100 μl of 0.06% hydrogen peroxide was added in each tube where whole stillage was treated with peroxidase. The treatment was carried out at 50° C. for 2 hours in a temperature-controlled water bath. After the treatment, a 100 μl treated whole stillage sample was taken and diluted with 4.9 ml deionized water prior to particle size analysis. The analysis was conducted the same way as in Example 2.

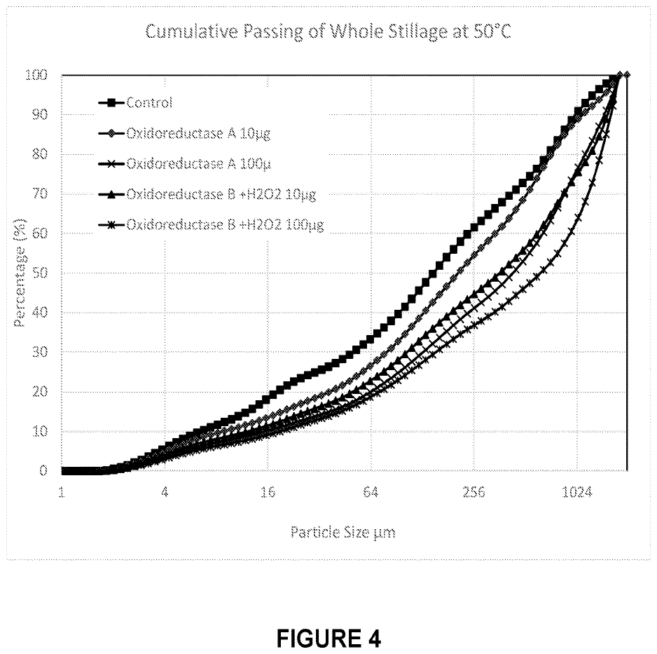

FIG. 4 shows the impact on cumulative passing of whole stillage teated with Oxidoreductase A and Oxidoreductase B at 50° C. Both oxidoreductases have shifted the particle size distribution towards larger diameter particle sizes. The larger dose of 100 μg/gDS produced a more pronounced increase of particle size than the lower dose of 10 μg/gDS, regardless of the oxidoreductase.

The impact of particle diameter by oxidoreductase treatment is shown in Table 3 below. The particles sized at or less than a specific diameter (μm) are shown at various levels of particle volume percentage. At 50% particle volume, no enzyme control and 10 μg Oxidoreductase A treated whole stillages have particle size s 162.8 μm and ≤227.5 μm, respectively. This indicates a greater quantity of smaller sized particles in the control sample without oxidoreductases compared to the 10 μg Oxidoreductase A-treated sample. In other words, 10 μg Oxidoreductase A treatment reduced smaller the volume % of smaller sized particles and increased the volume % of larger sized particles in the whole stillage.

TABLE 3
Impact of particle diameter by oxidoreductase treatment at 50° C.

| | Particle Diameter (≤um) | | | | |
|---|---|---|---|---|---|
| | | | | Oxidoreductase | Oxidoreducctase |
| Volume | | Oxidoreductase | Oxidoreductase | B | B |
| % | Control | A 10 μg | A 100 μg | 10 ug + H₂O₂ | 100 ug + H₂O₂ |
| 10 | 7.138 | 9.663 | 16.81 | 13.44 | 20.68 |
| 25 | 33.52 | 62.68 | 103 | 82.89 | 118.1 |
| 50 | 162.8 | 227.5 | 474.6 | 397.7 | 687.7 |
| 75 | 613.2 | 677.3 | 1091 | 1121 | 1429 |
| 90 | 1105 | 1209 | 1622 | 1692 | 1772 |

### Example 4: Increase Particle Size by Treating Whole Stillage with Oxidoreductases at 35° C.

The whole stillage from Example 2 was used in this example at 10 g/tube. Oxidoreductase A and Oxidoreductase B from Example 2 were used in this example and dosed at 10 μg/gDS. A 100 μl of 0.06% hydrogen peroxide was added with one peroxidase treatment. The treatment was carried out at 35° C. for 16 hours in a temperature-controlled water bath. After the treatment, the sample preparation and particle size analysis were conducted the same way as in Example 2.

The impact on particle size distribution by oxidoreductase treatment is shown in Table 4 below. The particle sized at or less than a specific diameter (μm) are shown at various levels of particle volume percentage. At 50% particle volume, no enzyme control and 10 μg Oxidoreductase A-treated whole stillages have particle size s 763.6 μm and ≤843.8 μm, respectively. This indicates a greater quantity of smaller sized particles in the control sample without enzymes than the 10 μg Oxidoreductase A-treated sample. In other words, 10 μg Oxidoreductase A treatment reduced the volume % of smaller sized particles and increased the volume % of larger sized particles. Similarly, Oxidoreductase B-treated samples decreased the volume % of smaller sized particles and increased the volume % of larger sized particles in the whole stillage.

TABLE 4
Impact of particle diameter by laccase and peroxidase treatment at 35° C.

| | Particle Diameter (um<) | | | |
|---|---|---|---|---|
| | No enzyme | | Peroxidase 10 μg + | |
| Volume % | Control | Laccase 10 μg | H202 | Peroxidase 10 μg |
| 10 | 30.27 | 41.41 | 50.04 | 57.61 |
| 25 | 206.1 | 286.8 | 385.5 | 470.2 |
| 50 | 763.6 | 843.8 | 823.9 | 887.7 |
| 75 | 1206 | 1259 | 1096 | 1291 |
| 90 | 1605 | 1648 | 1566 | 1708 |

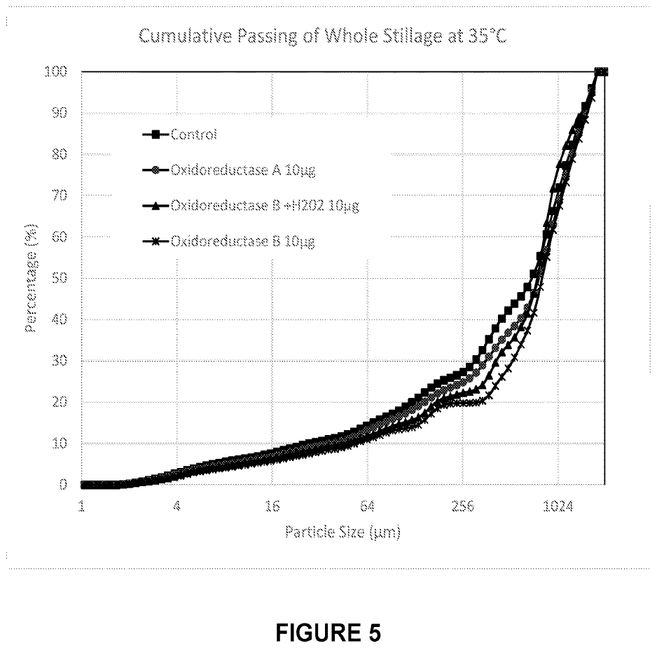

FIG. 5 shows the impact on cumulative passing of whole stillage treated with Oxidoreductase A and B treatment at 35° C. Oxidoreductase treatments have shifted the particle size towards larger diameter. The increase of particle size is smaller compared to treatment at 65° C. in Example 2, likely due to the relatively lower activity of the oxidoreductases at 35° C.

1. A process for reducing the viscosity of syrup, the process comprising:
(a) liquefying a starch-containing material with a thermostable alpha-amylase at a temperature above the initial gelatinization temperature of the starch-containing material to produce a dextrin;
(b) saccharifying the dextrin with a glucoamylase to produce a fermentable sugar;
(c) fermenting the sugar with a fermenting organism to produce a beer comprising the fermentation product;
(d) distilling the beer to recover the fermentation product and produce a whole stillage;
(e) processing the whole stillage to produce a thin stillage; and
(f) evaporating the thin stillage to produce a syrup;
wherein an oxidoreductase is added after the fermenting step to decrease the quantity of particles in the thin stillage by increasing the size of particles in the whole stillage, thereby reducing the viscosity of syrup produced by evaporating the thin stillage.

2. (canceled)

3. The process of claim 1, wherein the oxidoreductase is added to a beer well containing the beer prior to the distilling step.

4. The process of claim 1, wherein the oxidoreductase is added during the distilling step.

5. The process of claim 1, wherein the oxidoreductase is added to the whole stillage before the processing step.

6. (canceled)

7. (canceled)

8. The process of claim 1, wherein the oxidoreductase is added to the whole stillage and incubated for a period of time before the whole stillage is subjected to the separation step.

9. (canceled)

10. The process of claim 1, wherein the cumulative passing is reduced by a volume of from 10% to 80% for particles having a size ranging from 100 μm to 1000 μm.

11. The process of claim 1, wherein the cumulative passing of particles having a diameter of 400 μm is decreased by 10 volume % to 80 volume %.

12. The process of claim 1, wherein the oxidoreductase is a laccase.

13. The process of claim 1, wherein the oxidoreductase is a peroxidase.

14. The process of claim 1, wherein the saccharifying and fermenting steps are performed simultaneously.

15. (canceled)

16. (canceled)

17. (canceled)

18. A process for reducing viscosity of syrup in a producing a fermentation product from a starch-containing material, the process comprising:
(a) saccharifying a starch-containing material at a temperature below the initial gelatinization temperature of the starch with an alpha-amylase and a glucoamylase to produce fermentable a sugar;
(b) fermenting the sugar with a fermenting organism to produce a beer comprising the fermentation product;
(c) distilling the beer to recover the fermentation product and to produce a whole stillage;
(d) processing the whole stillage to produce a thin stillage; and
(e) evaporating the thin stillage to produce a syrup;
wherein an oxidoreductase is added after the fermenting step to decrease the quantity of particles in the thin stillage by increasing the size of particles in the whole stillage, thereby reducing the viscosity of syrup produced by evaporating the syrup.

19. The process of claim 18, wherein the oxidoreductase is added to a beer well containing the beer prior to the distilling step.

20. The process of claim 18, wherein the oxidoreductase is added during the distilling step.

21. The process of claim 18, wherein the oxidoreductase is added to the whole stillage before the processing step.

22. The process of claim 18, wherein the oxidoreductase is added to the whole stillage and incubated for a period of time before the whole stillage is subjected to the separation step.

23. The process of claim 18, wherein the cumulative passing is reduced by a volume of from 10% to 80% for particles having a size ranging from 100 μm to 1000 μm.

24. The process of claim 18, wherein the cumulative passing of particles having a diameter of 400 μm is decreased by 10 volume % to 80 volume %.

25. The process of claim 18, wherein the oxidoreductase is a laccase.

26. The process of claim 18, wherein the oxidoreductase is a peroxidase.

27. The process of claim 18, wherein the saccharifying and fermenting steps are performed simultaneously.


SEQUENCE LISTING (6 sequences)

SEQ ID NO: 1 - protein, 573 aa, Thermothelomyces thermophilus
QQSCNTPSNRACWTDGYDINTDYEVDSPDTGVVRPYTLTLTEVDNWTGPDGVVKEKVMLVNNSIIGPTIFADWGDTIQVTVINNLETNGTSIHWHGLHQKGTNLHDGANGITECPIPPKGGRKVYRFKAQQYGTSWYHSHFSAQYGNGVVGAIQINGPASLPYDTDLGVFPISDYYYSSADELVELTKNSGAPFSDNVLFNGTAKHPETGEGEYANVTLTPGRRHRLRLINTSVENHFQVSLVNHTMTIIAADMVPVNAMTVDSLFLGVGQRYDVVIEASRTPGNYWFNVTFGGGLLCGGSRNPYPAAIFHYAGAPGGPPTDEGKAPVDHNCLDLPNLKPVVARDVPLSGFAKRPDNTLDVTLDTTGTPLFVWKVNGSAINIDWGRPVVDYVLTQNTSFPPGYNIVEVNGADQWSYWLIENDPGAPFTLPHPMHLHGHDFYVLGRSPDESPASNERHVFDPARDAGLLSGANPVRRDVTMLPAFGWVVLAFRADNPGAWLFHCHIAWHVSGGLGVVYLERADDLRGAVSDADADDLDRLCADWRRYWPTNPYPKSDSGLKHRWVEEGEWLVKA

SEQ ID NO: 2 - protein, 521 aa, Coprinopsis cinerea
QIVNSVDTMTLTNANVSPDGFTRAGILVNGVHGPLIRGGKNDNFELNVVNDLDNPTMLRPTSIHWHGLFQRGTNWADGADGVNQCPISPGHAFLYKFTPAGHAGTFWYHSHFGTQYCDGLRGPMVIYDDNDPHAALYDEDDENTIITLADWYHIPAPSIQGAAQPDATLINGKGRYVGGPAAELSIVNVEQGKKYRMRLISLSCDPNWQFSIDGHELTIIEVDGQLTEPHTVDRLQIFTGQRYSFVLDANQPVDNYWIRAQPNKGRNGLAGTFANGVNSAILRYAGAANADPTTSANPNPAQLNEADLHALIDPAAPGIPTPGAADVNLRFQLGFSGGRFTINGTAYESPSVPTLLQIMSGAQSANDLLPAGSVYELPRNQVVELVVPAGVLGGPHPFHLHGHAFSVVRSAGSSTYNFVNPVKRDVVSLGVTGDEVTIRFVTDNPGPWFFHCHIEFHLMNGLAIVFAEDMANTVDANNPPVEWAQLCEIYDDLPPEATSIQTVVRRAEPTGFSAKFRREGL

SEQ ID NO: 3 - protein, 499 aa, Polyporus pinsitus
GIGPVADLTITNAAVSPDGFSRQAVVVNGGTPGPLITGNMGDRFQLNVIDNLTNHTMLKSTSIHWHGFFQKGTNWADGPAFINQCPISSGHSFLYDFQVPDQAGTFWYHSHLSTQYCDGLRGPFVVYDPNDPAADLYDVDNDDTVITLVDWYHVAAKLGPAFPLGADATLINGKGRSPSTTTADLSVISVTPGKRYRFRLVSLSCDPNYTFSIDGHNMTIIETDSINTAPLVVDSIQIFAAQRYSFVLEANQAVDNYWIRANPNFGNVGFTGGINSAILRYDGAAAVEPTTTQTTSTAPLNEVNLHPLVTTAVPGSPVAGGVDLAINMAFNFNGTNFFINGASFTPPTVPVLLQIISGAQNAQDLLPSGSVYSLPSNADIEISFPATAAAPGAPHPFHLHGHAFAVVRSAGSTVYNYDNPIFRDVVSTGTPAAGDNVTIRFRTDNPGPWFLHCHIDFHLEAGFAVVFAEDIPDVASANPVPQAWSDLCPTYDALDPSDQ

SEQ ID NO: 4 - protein, 345 aa, Coprinopsis cinerea
LPQGPGGGGSVTCPGGQSTSNSQCCVWFDVLDDLQTNFYQGSKCESPVRKILRIVFHDAIGFSPALTAAGQFGGGGADGSIIAHSNIELAFPANGGLTDTVEALRAVGINHGVSFGDLIQFATAVGMSNCPGSPRLEFLTGRSNSSQPSPPSLIPGPGNTVTAILDRMGDAGFSPDEVVDLLAAHSLASQEGLNSAIFRSPLDSTPQVFDTQFYIETLLKGTTQPGPSLGFAEELSPFPGEFRMRSDALLARDSRTACRWQSMTSSNEVMGQRYRAAMAKMSVLGFDRNALTDCSDVIPSAVSNNAAPVIPGGLTVDDIEVSCPSEPFPEIATASGPLPSLAPAP

SEQ ID NO: 5 - protein, 326 aa, Glycine max
QLTPTFYRETCPNLFPIVFGVIFDASFTDPRIGASLMRLHFHDCFVQGCDGSVLLNNTDTIESEQDALPNINSIRGLDVVNDIKTAVENSCPDTVSCADILAIAAEIASVLGGGPGWPVPLGRRDSLTANRTLANQNLPAPFFNLTQLKASFAVQGLNTLDLVTLSGGHTFGRARCSTFINRLYNFSNTGNPDPTLNTTYLEVLRARCPQNATGDNLTNLDLSTPDQFDNRYYSNLLQLNGLLQSDQELFSTPGADTIPIVNSFSSNQNTFFSNFRVSMIKMGNIGVLTGDEGEIRLQCNFVNGDSFGLASVASKDAKQKLVAQSK

SEQ ID NO: 6 - protein, 323 aa, Armoracia rusticana
QLTPTFIDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSLLHDMVEVVDFVSSM